(19) United States
(12) Patent Application Publication
(10) Pub. No.: US 2007/0042043 A1
(43) Pub. Date: Feb. 22, 2007

(54) Pellet formulations of acid-labile benzimidazonle compounds

(76) Inventors: Luis Carvajal Martin, Madrid (ES); Juan Carlos Asensio Asensio, Zaragoza (ES); Francisco Javier Sevilla Tirado, Zaragoza (ES)

Correspondence Address: HENSLEY KIM & EDGINGTON, LLC, 1660 LINCOLN STREET, SUITE 3050, DENVER, CO, 80264, US

(21) Appl. No.: 10/554,727

(22) PCT Filed: Apr. 27, 2004
(86) PCT No.: PCT/EP04/50618

§ 371 Date: Oct. 28, 2005

(30) Foreign Application Priority Data
Apr. 29, 2003 (ES) P200300976

(51) Int. Cl. A61K 31/4439; A61K 9/26; A61K 9/14

(52) U.S. Cl. 424/470; 514/338; 424/489

(57) ABSTRACT

They comprise insert granules of sugar/starch which are: initially coated with a non-alkaline active layer having the benzimidazole compound (omeprazole, lansoprazole, pantoprazole, rabeprazole, etc.), sodium and/potassium salts of acids of formula R—O—SO₃H wherein R is an alkyl radical of a (C₆-C₂₀)-fatty acid (preferably sodium lauryl sulfate), (C₆-C₂₀)-fatty acids (preferably oleic acid), sodium and/or potassium salts of (C₆-C₂₀)-fatty acids (preferably potassium oleate), sodium carboxymethyl starch and polyvinylpyrrolidone; secondly coated with a non-alkaline barrier layer having hydroxypropylmethylcellulose; and finally coated with an enteric layer. The preferred molar ratio (sodium lauryl sulfate):(oleic acid+potassium oleate) is between 4:1 and 6:1. All coatings are done with aqueous solutions, suspensions or dispersions at a relatively high temperature, and all dryings are done at a relatively low temperature and for a relatively short time. They are stable over time and useful for oral administration.

The present invention relates to new pellet formulations for oral administration of known acid labile benzimidazole compounds, and to a preparation process thereof.

### BACKGROUND ART

Benzimidazole compounds of formula (I), wherein R1 is selected from the group consisting of hydrogen, methoxy and difluoromethoxy; R2 is selected from the group consisting of methyl and methoxy; R3 is selected from the group consisting of methoxy, 2,2,2-trifluoroethoxy and 3-methoxypropoxy; R4 is selected from the group consisting of hydrogen and methyl are a group of pharmaceutical active ingredients known to be useful for the treatment of gastrointestinal diseases and/or disorders. This group include commercial pharmaceutical active substances such as omeprazole (wherein R1=R3=methoxy; R2=R4=methyl), lansoprazole (wherein R1=R4=hydrogen; R2=methyl; R3=2,2,2-trifluoroethoxy), pantoprazole (wherein R1=difluoromethoxy; R2=R3=methoxy; R4=hydrogen) and rabeprazole (wherein R1=R4=hydrogen; R2=methyl; R3=3-methoxypropoxy). Hereinafter any of these compounds in isolation, or any selected group of them, is referred to simply as “benzimidazole compound”.

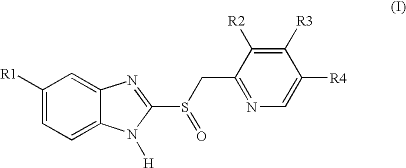

It is well known that the benzimidazole compound has a poor stability over time. In the solid state it is susceptible to moisture, light and heat, and in aqueous solution or suspension the stability decreases with decreasing pH. Its degradation becomes apparent through discoloration and it is catalyzed by acid substances. Since the compound is primarily absorbed in the intestine, it is obvious that any oral pharmaceutical formulation of the benzimidazole compound requires some kind of outer enteric coating in order to protect the active ingredient from the acid of the stomach. As conventional enteric coatings are acidic, the need of protecting the pharmaceutical active ingredient from the enteric coating became an evident problem, as soon as the acid labile properties of the benzimidazole compound were known. The use of pellet formulations with one or more barrier layers to protect the pharmaceutical active ingredient from the enteric layer has been the most frequently chosen solution to this problem. However, the choice of a particular combination of core and barrier layers in the pellet formulation, in order to provide both efficient manufacturing processes while maintaining a high degree of stability, remains a delicate matter. This is illustrated by the fact that some different approaches to the problem and several different solutions have been reported in the art.

One of the approaches to the problem involves the use of solid alkaline reactive substances in contact with the benzimidazole compound, in such a way that an alkaline pH is obtained when water is absorbed or added to the particles. In EP 244.380-A and EP 247.983-A (both from Hässle, now Astra-Zeneca) pellet formulations containing active cores of the benzimidazole compound with solid alkaline reactive substances were prepared via the classical pellet manufacturing method of mixture-extrusion-spherification. The solid alkaline reactive substances mentioned in the examples are aluminium hydroxide, magnesium hydroxide, magnesium carbonate, sodium carbonate, magnesium oxide, disodium hydrogenphosphate, sodium dihydrogenphosphate, synthetic hydrotalcite and mixtures thereof; other solid alkaline reactive substances were suggested as potentially useful. In preferred embodiments the same solid alkaline reactive substances are also included in the barrier layer separating the active layer from the outer enteric one.

Most of pellet formulations of benzimidazole compound described in the art, and in particular those mentioned below, do not use the mixture-extrusion-spherification method of pellet manufacturing, but the alternative one comprising: coating inert nuclei of sucrose/starch with an active layer containing the benzimidazole compound; drying; coating with a barrier layer; drying; coating with a enteric layer, and drying. Nevertheless, in some of the below-mentioned documents, pellet manufacturing method of mixture-extrusion-spherification or tablet manufacturing methods are mentioned as well.

In pellet formulations described in EP 237.200-A (Takeda) the solid alkaline reactive substance is a basic inorganic salt of magnesium or calcium in even contact with the benzimidazole compound. In EP 277.741-A (Takeda) the improvement of having low substituted hydroxypropyl cellulose in the active layer is described.

In all of the above-mentioned documents there are always solid alkaline reactive substances in contact with the benzimidazole compound. However, a different approach to the solution of the same problem involves pellet formulations which do not have any solid alkailne reactive substance in contact with the benzimidazole compound, and frequently have no solid alkaline reactive substances in the barrier layer.

In pellet formulations described in WO 9938511-A and WO 0071121-A (both from Ethypharm) the non-alkaline reactive substance is a hydrophobic substance, preferably a glyceride or a silicone oil. Another important technical feature of these pellet formulations is that they do not have any anionic surfactant (e.g. sodium lauryl sulfate, which is explicitly excluded).

In pellet formulations described in WO 9623500-A (Esteve), the non-alkaline reactive substance is talc, both in the active layer and in the barrier layer. Another important technical feature of these pellets formulations is that they include hydroxypropylmethylcellulose in both the active layer and in the barrier layer.

In pellet formulations described in WO 9637195-A (Mepha) the non-alkaline reactive substance is titanium dioxide, both in the active layer and in the barrier layer. The use of hydroxypropylmethylcellulose in the active layer is also described.

In pellet formulations described in WO 9325204-A (Ethypharm) the active layer contains sodium lauryl sulfate and sodium carboxymethyl starch (Explotab®); but there are no fatty acids of fatty acid salts anywhere in the pellet, and the non-alkaline reactive substance is mannitol. Another important feature is that both the active layer and the barrier are applied with an alcoholic solution (95° ethanol, 80%; water, 20%), not with an aqueous solution or suspension.

Despite the relative high number of pellet formulations of benzimidazole compounds known in the art, there is still an active research in this field because there is still area for improvement in industrial preparation parameters (e.g. higher temperatures and/or shorter times), costs (lower amounts and/or lower prices of excipients), gastric resistance, intestine absorption (bioavailability), and/or storage stability. The present invention provides an alternative solution to those known in the art, which involves several advantages compared with some commercial formulations, and especially compared with those which are the-closest when considering the technical point of view.

### SUMMARY OF THE INVENTION

An aspect of the present invention relates to the provision of a pharmaceutical pellet formulation for oral administration of a benzimidazole compound of formula (I), or of a stereoisomer thereof, wherein R1 is selected from the group consisting of hydrogen, methoxy and difluoromethoxy; R2 is selected from the group consisting of methyl and methoxy; R3 is selected from the group consisting of methoxy, 2,2,2-trifluoroethoxy and 3-methoxypropoxy; R4 is selected from the group consisting of hydrogen and methyl; the pellet formulation comprising inert granules which are: (a) initially coated with a non-alkaline reactive active layer comprising a benzimidazole compound (I), pharmaceutically acceptable sodium and/or potassium salts of acids of formula R—O—SO₃H wherein R is an alkyl radical of a (C₆-C₂₀)-fatty acid, non-alkaline pharmaceutically acceptable disintegrants, non-alkaline pharmaceutically acceptable binders, a substantial amount of (C₆-C₂₀)-fatty acids and a substantial amount of sodium and/or potassium salts of (C₆-C₂₀)-fatty acids, these two amounts being in a molar ratio [acids]:[salts] between 1:4 and 4:1; (b) secondly coated with a non-alkaline reactive barrier layer comprising one or more pharmaceutically acceptable coating excipients; and (c) finally coated with an enteric coating layer. In a preferred embodiment, the molar ratio [salts of acids of formula R—O—SO₃H]:[(C₆-C₂₀)-fatty acids+salts of (C₆-C₂₀)-fatty acids] is between 4:1 and 6:1. In a still more preferred embodiment, the salts of acids R—O—SO₃H comprise sodium lauryl sulfate, the (C₆-C₂₀)-fatty acids comprise oleic acid, and the salts of (C₆-C₂₀)fatty acids comprise potassium oleate. Some pharmaceutical formulations of benzimidazole compounds of formula (I) with fatty acid salts, particularly with sodium oleate, are known in the art (cf. EP 645.140-A, Takeda), but they are only for rectal (not oral) administration, and they are not of the coated pellet type.

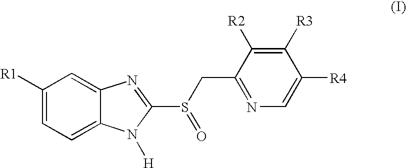

The term “(C₆-C₂₀)-fatty acids” includes any of the known saturated and unsaturated fatty acids of 6 to 20 carbon atoms, such as caproic acid, caprylic acid, capric acid, lauric acid, oleic acid, linolic acid, linolenic acid, arachidonic acid, myristic acid, palmitic acid, stearic acid, etc. Inventors have surprisingly found that the combination of the salts of acids R—O—SO₃H and the buffer-like mixture of (C₆-C₂₀)-fatty acids and their salts, improves the protection of the benzimidazole compound from water residues, the acidic enteric layer and the environment, without the need of any solid alkaline-reacting substance. In addition, the dissolution of the benzimidazole compound in the intestine is highly favored by the surfactant properties of both types of salts.

The pharmaceutically acceptable disintegrant in the active layer contribute to the rapid disintegration of the pellets in the intestine. Any non-alkaline disintegrant known in the art may be used, such as sodium carboxymethyl starch (Explotab®), crosspovidone or croscarmellose sodium. In a preferred embodiment, some amount of disintegrant is also included in the barrier layer. In a still more preferred embodiment, sodium carboxymethyl starch is the disintegrant of choice.

Spraying of the active layer over the inert nuclei of sugar/starch is highly favored by the addition of a non-alkaline binder, which may be selected from those known in the art, such as hydroxypropyl cellulose, hydroxypropylmethylcellulose, polyvinylpyrrolidone, etc. Polyvinylpyrrolidone is a preferred binder.

The coating agent (binder) in the barrier layer may be selected from the group of polyvinylpyrrolidone, hydroxypropyl cellulose and hydroxypropylmethylcellulose, the latter being a preferred one.

Ingredients of the enteric layer may be selected from any of those well known by persons skilled in the art, the combination of triethyl citrate, poly(methacrylic acid, ethyl acrylate) and titanium dioxide being a preferred one. In a more preferred embodiment, some triethyl citrate is also included in the barrier layer.

In the particular embodiments illustrated in the accompanying examples, benzimidazole compound of formula (I) are omeprazole or lansoprazole, although the pellet formulations of the invention may also be used with other benzimidazole compounds of formula (I), such as pantoprazole or rabeprazole.

Another aspect of the invention refers to a preparation process of a pharmaceutical pellet formulation for oral administration of a benzimidazole compound of formula (I), comprising the steps of (i) starting with inert granules; (ii) coating initially with an aqueous suspension comprising a benzimidazole compound of formula (I), pharmaceutically acceptable sodium and/or potassium salts of acids of formula R—O—SO₃H wherein R is an alkyl radical of a (C₆-C₂₀)-fatty acid, non-alkaline pharmaceutically acceptable disintegrants, non-alkaline pharmaceutically acceptable binders, a substantial amount of (C₆-C₂₀)-fatty acids and a substantial amount of sodium and/or potassium salts of (C₆-C₂₀)-fatty acids, these two amounts being in a molar ratio [acids]:[salts] between 1:4 and 4:1; (iii) drying to yield one-layer-coated granules; (iv) coating secondly with an aqueous suspension comprising one or more non-alkaline reactive pharmaceutically acceptable coating excipients; (v) drying to yield two-layer-coated granules; (vi) coating thirdly with an aqueous suspension comprising enteric coating agents; and (vii) finally drying to yield enteric three-layer-coated granules. Coated granules obtained after drying of steps (iii), (v) and (vii) are non-alkaline reactive when dispersed in water. As an optional precautionary measure, coating steps (ii) and (iv) may be done with the help of the necessary amounts of a 30% aqueous ammonia solution to get a pH of around 8.5. In this case, drying steps (iii) and (v) completely remove ammonia gas from the pellet.

Pellet formulations of the present invention fulfill all the pharmaceutical requirements, which are well-known to persons skilled in the art, such as level of impurities (total lower than 1%), gastroresistance (greater than 90% at pH 1.2 for 2 h) and bioavailability (dissolution and absorption in the intestine). In addition, they show significant advantages with respect to stability over time (e.g. in standard accelerated tests at 40° C. and 75% relative humidity, for 6 months) over other pellet formulations known in the art (e.g. the two of Table 1 in WO 9623500-A).

The loading of active ingredient of the pellets of the present invention can be as high as 10% by weight, which is higher than the loading of some pellet formulation known in the art (e.g. the 8.4% of the one described in WO 9325204-A). This feature represents both a shorter manufacturing time and an economic saving in excipients. Another advantage of the present invention over that of WO 9325204-A is the use of water as the only solvent, thus avoiding the more problematic use of ethanol or other organic solvents.

The preparation process of the pellet formulations of the present invention allows a first active coating at a relatively high temperature of granules (from about 50° C. to about 70° C.), substantially higher than the temperature corresponding to processes known in the art (e.g. 40° C. in EP 237.200-A). It also allows drying at relatively low temperatures (25-35° C.) and/or for short times (20 min), all of which represent manufacturing advantages over other processes known in the art (e.g. 40° C. for 16 h in EP 237.200-A; 50° C. for 4 h in WO 0071121-A).

Throughout the description and claims the word “comprise” and its variations are not intended to exclude other features, components, or steps. The disclosure in the abstract accompanying this application is incorporated herein as reference. Additional objects, advantages and features of the invention will be set forth in part in the description, and in part will become apparent to those skilled in the art upon examination of the description or may be learned by practice of the invention. The following examples are provided by way of illustration, and they are not intended to be limiting the present invention.

### DETAILED DESCRIPTION OF PARTICULAR EMBODIMENTS

### Example 1

### Industrial Preparation Process of Pellet Formulations of Omeprazole and Lansoprazole

Batches of 600 kg of a pellet formulation of the benzimidazole compound (omeprazole or lansoprazole) were prepared in a standard film-coating machine according to the following steps:
Step 1: Inert granules (302.40 kg, 0.7-0.9 mm diameter) of sucrose (80%) and starch (20%) were introduced In the machine and warmed to 30-35° C.
Step 2: An aqueous solution of sodium carboxymethyl starch (Explotab®; 11.34 kg), polyvinylpyrrolidone (29.925 kg) and sodium lauryl sulfate (18.27 kg) was prepared by addition of the ingredients to enough water.
Step 3: In a separate vessel, an aqueous solution of potassium oleate and oleic acid was prepared by dissolving 4.2525 kg of oleic acid and 0.214 kg of potassium hydroxide in water.
Step 4: The solution of Step 3 was added to the solution of Step 2 until an homogeneous solution was obtained.
Step 5: The required amount of benzimidazole compound (63.00 kg of omeprazole or lansoprazole) was added slowly to the solution of Step 4 to get a suspension, and maintained at approximately 10° C.
Step 6: The inert granules of Step 1 were sprayed with the suspension of Step 5, at 40-50° C., under a relative humidity lower than 12.5%.
Step 7: One-layer-coated granules obtained in Step 6 were dried at a temperature of 35-45° C., and relative humidity lower than 10.5%, for 20 min, yielding dry granules with less than 2% water which gave a pH around 7 when dispersed in water.
Step 8: Dried one-layer-coated granules obtained in Step 7 were sieved with a 0.99 mm sieve.
Step 9: Granules having passed the sieving of Step 8 were warmed to 30-35° C.
Step 10: An aqueous solution of hydroxypropylmethylcellulose (18.90 kg), sodium carboxymethyl starch (Explotab®; 1.89 kg) and triethyl citrate (1.89 kg) was prepared.
Step 11: Granules of Step 9 were sprayed with the solution of Step 10, at 40-50° C., under a relative humidity lower than 12.5%.
Step 12: Two-layer-coated granules of Step 11 were dried at 35-45° C. and relative humidity lower than 10.5%, for 20 min, yielding granules with less than 2% water which gave a pH around 7 when dispersed in water.
Step 13: Dried two-layer-coated granules obtained in Step 12 were sieved with a 1.18 mm sieve.
Step 14: Granules having passed the sieving of Step 13 were warmed to 30-32° C.
Step 15: An aqueous dispersion of poly(methacrylic acid, ethyl acrylate) 1:1 (Eudragit®; 129.185 kg) and triethyl citrate (12.92 kg) was prepared, keeping it cold and under constant stirring.
Step 16: An aqueous suspension of titanium dioxide (4.72 kg) was prepared.
Step 17: The aqueous suspension of Step 16 was added to the aqueous dispersion of Step 15 with continues stirring until total homogeneity was reached, and it was kept at a temperature lower than 10° C.
Step 18: Granules of Step 13 were sprayed with the dispersion of Step 17 at 30-35° C., under a relative humidity of less than 20%.
Step 19: Granules of Step 18 were dried at 25-35° C., under a relative humidity of less than 10.5%, for 20 min, yielding enteric-coated granules with less than 2% water.
Step 20: Enteric-coated granules of Step 19 were sieved with a 1.25 mm sieve and lubrified with talc (0.6 kg), yielding a pellet formulation with 105 mg of benzimidazole compound per gram.

As an extra precaution, in Steps 4 and 10 the minimum necessary amounts of 30% aqueous ammonia solution to get a pH of 8:5 were added, all ammonia being evaporated in drying Steps 7 and 12. Both omeprazole and lansoprazole pellet formulations thus obtained had less than 1% impurities and a gastroresistance greater than 90% at pH 1.2 for 2 h; and their 1% suspensions in water had pH lower than 7.0.

### Example 2

### Comparative Dissolution Tests

Pellet formulations with the same amount of omeprazole or lansoprazole prepared according to the process of Example 1, and commercially available pellet formulations with the same amounts of the same pharmaceutical active ingredient, were submitted to respective comparative dissolution tests in aqueous solutions of pH 6.8, in the same apparatus, stirring at the same speed (100 rpm), at the same temperature (37° C.), and for the same times.

A lansoprazole pellet formulation prepared according to Example 1 showed similar dissolution profiles than commercial Opiren® (lansoprazole from Almirall Prodesfarma, under license of Takeda). Thus, after 15 min, percentages of lansoprazole dissolved were: Opiren®, 58.4%; present invention, 69.3%. And after 30 min, percentages were: 71.3% and 86.5%, respectively. All pellets had been obtained by coating of inert nuclei, and those under license from Takeda presumably had been obtained with the process described in the above-mentioned documents EP 237.200-A and/or EP 277.741-A.

An omeprazole pellet formulation prepared according to Example 1 showed similar dissolution profiles to commercial Losec® (omeprazole from Astra-Zeneca), similar to commercial Mopral® (omeprazole from Astra-Zeneca), and faster than an omeprazole pellet formulation prepared according to the Ethypharm WO 9325204-A process. Thus, after 15 min, percentages of omeprazole dissolved were: Losec®, 89.1%;, Mopral®, 89.0%; Ethypharm process, 60.1%; present invention, 93.7%. And after 30 min, percentages were: 85.0%, 90.9%, 80.0% and 92.3%, respectively.

1. A pharmaceutical pellet formulation for oral administration of a benzimidazole compound of formula (I), or a stereoisomer thereof, 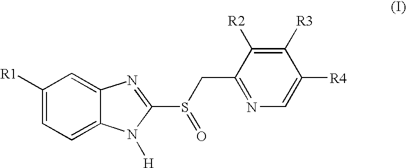
wherein R1 is selected from the group consisting of hydrogen, methoxy and difluoromethoxy; R2 is selected from the group consisting of methyl and methoxy; R3 is selected from the group consisting of methoxy, 2,2,2-trifluoroethoxy and 3-methoxypropoxy; R4 is selected from the group consisting of hydrogen and methyl; said pellet formulation comprising inert granules which are:
(a) initially coated with a non-alkaline reactive active layer comprising a benzimidazole compound (I), pharmaceutically acceptable sodium and/or potassium salts of acids of formula R—O—SO₃H wherein R is an alkyl radical of a (C₆-C₂₀)-fatty acid, non-alkaline pharmaceutically acceptable disintegrants and non-alkaline pharmaceutical binders;
(b) secondly coated with a non-alkaline reactive barrier layer comprising one or more pharmaceutically acceptable coating excipients; and
(c) finally coated with a pharmaceutically acceptable enteric coating layer;
whereby the active layer also has a substantial amount of (C₆-C₂₀)-fatty acids and a substantial amount of sodium and/or potassium salts of (C₆-C₂₀)-fatty acids, these two amounts being in a molar ratio [acids]:[salts] between 1:4 and 4:1.

2. The pharmaceutical pellet formulation according to claim 1, wherein the molar ratio [acids]:[salts] is a molar ratio [salts of acids of formula R—O—SO₃H]:[(C₆-C₂₀)-fatty acids+salts of (C₆-C₂₀)-fatty acids] which is between 4:1 and 6:1.

3. The pharmaceutical pellet formulation according to claim 1, wherein the salts of acids of formula R—O—SO₃H comprise sodium lauryl sulfate, the (C₆-C₂₀)-fatty acids comprise oleic acid, and the salts of (C₆-C₂₀)-fatty acids comprise potassium oleate.

4. The pharmaceutical pellet formulation according to claim 1, wherein the active layer comprises sodium carboxymethyl starch as disintegrant.

5. The pharmaceutical pellet formulation according to claim 1, wherein the active layer comprises polyvinylpyrrolidone as binder.

6. The pharmaceutical pellet formulation according to claim 1, wherein the pharmaceutically acceptable coating agents in the barrier layer are selected from the group consisting of polyvinylpyrrolidone, hydroxypropyl cellulose, hydroxypropylmethylcellulose, and mixtures thereof.

7. The pharmaceutical pellet formulation according to claim 1, wherein the enteric layer comprises triethyl citrate, poly(methacrylic acid, ethyl acrylate) and titanium dioxide.

8. The pharmaceutical pellet formulation according to claim 1, wherein the benzimidazole compound is omeprazole.

9. The pharmaceutical pellet formulation according to claim 1, wherein the benzimidazole compound is lansoprazole.

10. A preparation process of the pharmaceutical pellet formulation as defined in claim 1, comprising the steps of: (i) starting with inert granules; (ii) coating initially with an aqueous suspension comprising a benzimidazole compound of formula (I), pharmaceutically acceptable sodium and/or potassium salts of acids of formula R—O—SO₃H wherein R is an alkyl radical of a (C₆-C₂₀)-fatty acid, non-alkaline pharmaceutically acceptable disintegrants, non-alkaline pharmaceutically acceptable binders, a substantial amount of (C₆-C₂₀)-fatty acids and a substantial amount of sodium and/or potassium salts of (C₆-C₂₀)-fatty acids, these two amounts being in a molar ratio [acids]:[salts] between 1:4 and 4:1, (iii) drying to yield one-layer-coated granules; (iv) coating secondly with an aqueous suspension comprising one or more non-alkaline reactive pharmaceutically acceptable coating excipients; (v) drying to yield two-layer-coated granules; (vi) coating thirdly with an aqueous suspension comprising pharmaceutically acceptable enteric coating agents; and (vii) finally drying to yield enteric three-layer-coated granules; wherein the corresponding ingredients are respectively as defined in claim 1.

11. A method for the preparation of a pharmaceutical pellet formulation for oral administration of a benzimidazole compound of formula (I), or a stereoisomer thereof, 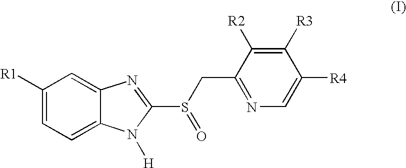
wherein R1 is selected from the group consisting of hydrogen, methoxy and difluoromethoxy; R2 is selected from the group consisting of methyl and methoxy; R3 is selected from the group consisting of methoxy, 2,2,2-trifluoroethoxy and 3-methoxypropoxy; R4 is selected from the group consisting of hydrogen and methyl; the method for the preparation of the pellet formulation comprising the steps of:
(i) starting with an inert granule;
(ii) coating the inert granule initially with an aqueous suspension comprising a benzimidazole compound of formula (I), pharmaceutically acceptable sodium and/or potassium salts of acids of formula R—O—SO₃H wherein R is an alkyl radical of a (C₆-C₂₀)-fatty acid, non-alkaline pharmaceutically acceptable disintegrants, non-alkaline pharmaceutically acceptable binders, a substantial amount of (C₆-C₂₀)-fatty acids and a substantial amount of sodium and/or potassium salts of (C₆-C₂₀)-fatty acids, these two amounts being in a molar ratio [acids]:[salts] between 1:4 and 4:1,
(iii) drying to yield a one-layer-coated granule;
(iv) coating the one-layer-coated granule secondly with an aqueous suspension comprising one or more non-alkaline reactive pharmaceutically acceptable coating excipients;
(v) drying to yield a two-layer-coated granule;
(vi) coating the two-layer-coated granule thirdly with an aqueous suspension comprising a pharmaceutically acceptable enteric coating agent; and
(vii) finally drying to yield an enteric three-layer-coated granule.

12. The method for the preparation of the pharmaceutical pellet formulation according to claim 11, wherein the molar ratio [acids]:[salts] is a molar ratio [salts of acids of formula R—O—SO₃H]:[(C₆-C₂₀)-fatty acids+salts of (C₆-C₂₀)-fatty acids] which is between 4:1 and 6:1.

13. The method for the preparation of the pharmaceutical pellet formulation according to claim 11, wherein the salts of acids of formula R—O—SO₃H comprise sodium lauryl sulfate, the (C₆-C₂₀)-fatty acids comprise oleic acid, and the salts of (C₆-C₂₀)-fatty acids comprise potassium oleate.

14. The method for the preparation of the pharmaceutical pellet formulation according to claim 11, wherein the active layer comprises sodium carboxymethyl starch as disintegrant.

15. The method for the preparation of the pharmaceutical pellet formulation according to claim 1, wherein the active layer comprises polyvinylpyrrolidone as binder.

16. The method for the preparation of the pharmaceutical pellet formulation according to claim 11, wherein the pharmaceutically acceptable coating agent in the barrier layer is selected from the group consisting of polyvinylpyrrolidone, hydroxypropyl cellulose, hydroxypropylmethylcellulose, and mixtures thereof.

17. The method for the preparation of the pharmaceutical pellet formulation according to claim 11, wherein the enteric layer comprises triethyl citrate, poly(methacrylic acid, ethyl acrylate) and titanium dioxide.

18. The method for the preparation of the pharmaceutical pellet formulation according to claim 11, wherein the benzimidazole compound is omeprazole.

19. The method for the preparation of the pharmaceutical pellet formulation according to claim 11, wherein the benzimidazole compound is lansoprazole.

20. A method for the use of a pharmaceutical pellet formulation of a benzimidazole compound of formula (I), or a stereoisomer thereof, 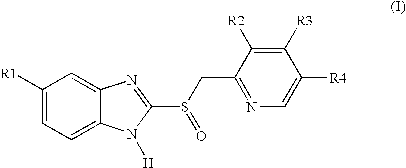
wherein R1 is selected from the group consisting of hydrogen, methoxy and difluoromethoxy; R2 is selected from the group consisting of methyl and methoxy; R3 is selected from the group consisting of methoxy, 2,2,2-trifluoroethoxy and 3-methoxypropoxy; R4 is selected from the group consisting of hydrogen and methyl; the pellet formulation including an inert granule that is triply-coated: (a) initially coated with a non-alkaline reactive active layer comprising a benzimidazole compound (I), pharmaceutically acceptable sodium and/or potassium salts of acids of formula R—O—SO₃H wherein R is an alkyl radical of a (C₆-C₂₀)-fatty acid, non-alkaline pharmaceutically acceptable disintegrants and non-alkaline pharmaceutical binders; (b) secondly coated with a non-alkaline reactive barrier layer comprising one or more pharmaceutically acceptable coating excipients; and (c) thirdly coated with a pharmaceutically acceptable enteric coating layer; wherein the active layer also has a substantial amount of (C₆-C₂₀)-fatty acids and a substantial amount of sodium and/or potassium salts of (C₆-C₂₀)-fatty acids, these two amounts being in a molar ratio [acids]:[salts] between 1:4 and 4:1;
the method of use of said pellet formulation comprising:
orally administering said pellet formulation to a recipient; and
providing for internal dissolution of the pellet formulation to deliver a pharmaceutically effective ingredient to the recipient.
